(19) United States
(12) Patent Application Publication
(10) Pub. No.: US 2025/0169492 A1
(43) Pub. Date: May 29, 2025

(54) Storage System for Biological Samples in Freezing Tanks in a Liquid/Gas Nitrogen Environment

(71) Applicant: Roee Sela, Sal'it (IL)

(72) Inventor: Roee Sela, Sal'it (IL)

(21) Appl. No.: 19/038,428

(22) Filed: Jan. 27, 2025

Related U.S. Application Data
(63) Continuation-in-part of application No. 17/481,015, filed on Sep. 21, 2021, now Pat. No. 12,233,421.
(60) Provisional application No. 63/080,940, filed on Sep. 21, 2020.

(51) Int. Cl. A01N 1/145; C12N 5/00

(52) U.S. Cl. CPC A01N 1/145; C12N 5/545

(57) ABSTRACT

The storage system is intended to provide users a device, that may securely hold biological samples within a canister in a space saving storage assembly. It is further an aim of the storage system to enable easy retrieval of the biological samples through a simple canister removal mechanism. Furthermore, the system includes a compact storage assembly that comprises multiple cells and canisters stacked together having efficient structural components that are suited for a typical storage tank for biological samples. Further improvements comprise a structural support for each canister, a corrugated panel structure forming a honeycomb canister organizational matrix, and a locking mechanism.

### FIELD OF THE INVENTION

The present invention relates generally to a space saving system for storing biological material in Nitrogen in a liquid/gaseous phase.

### BACKGROUND OF THE INVENTION

In a world where the storage of biological samples increases rapidly, there is a growing use of more and more storage spaces, and a significant increase in use of expensive materials used for this purpose. The need for more efficient storage of biological samples has arisen. Stored biological samples are often expensive and sensitive material. Therefore, there is a need for a construction that will allow robust and safe storage for the material inside the freezing tanks, while allowing fast and easy access to the biological samples.

An objective of the present invention is to provide users with a storage system, that may securely hold biological samples within a canister in a space saving storage assembly arranged in a honeycomb configuration within a storage tank. It is further an aim of the present invention to enable easy management, identification, and removal of biological samples. Furthermore, the present invention comprises a compact storage system that comprises multiple corrugated panels comprising columns to house canisters, a plurality of canisters, and integrated receptacles suited for a typical freezer tank for biological samples. Additionally, the present invention comprises a canister removal mechanism that enables easy and fast retrieval of a single canister from the frame.

### BRIEF DESCRIPTION OF THE DRAWINGS

FIG. 1 is a perspective view of the present invention.

FIG. 2 is a perspective view of the storage tank and frame of the present invention.

FIG. 3 is an exploded perspective view of the frame of the present invention.

FIG. 4 is a perspective view of one embodiment of a corrugated panel of the present invention.

FIG. 5 is a perspective view of an alternate embodiment of a corrugated panel of the present invention.

FIG. 6 is a perspective view of one embodiment of an end cap of the present invention.

FIG. 7 is a perspective view of the canister and receptacles of the present invention.

FIG. 8 is a perspective view of the canister of the present invention.

FIG. 9 is a front view of the guide axel of the present invention.

FIG. 10 is a detailed perspective view of the L-shaped tab and pull bar of the present invention.

FIG. 11 is a perspective view of the locking mechanism of the present invention.

FIG. 12 is a perspective view of the receptacle of the present invention.

FIG. 13 is a top view of the receptacle of the present invention.

FIG. 14 is a bottom view of the receptacle of the present invention.

FIG. 15 is a front perspective view of the sheet composing the receptacle of the present invention.

FIG. 16 is a perspective view of the canister removal mechanism of the present invention.

### DETAIL DESCRIPTIONS OF THE INVENTION

All illustrations of the drawings are for the purpose of describing selected versions of the present invention and are not intended to limit the scope of the present invention.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Additionally, it is important to note that each term used herein refers to that which an ordinary artisan would understand such term to mean based on the contextual use of such term herein. To the extent that the meaning of a term used herein-as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

Other technical advantages may become readily apparent to one of ordinary skill in the art after review of the following figures and description. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Unless otherwise indicated, the drawings are intended to be read together with the specification, and are to be considered a portion of the entire written description of this invention. As used in the following description, the terms “horizontal”, “vertical”, “left”, “right”, “up”, “down” and the like, as well as adjectival and adverbial derivatives thereof (e.g., “horizontally”, “rightwardly”, “upwardly”, “radially”, etc.), simply refer to the orientation of the illustrated structure as the particular drawing figure faces the reader. Similarly, the terms “inwardly,” “outwardly” and “radially” generally refer to the orientation of a surface relative to its axis of elongation, or axis of rotation, as appropriate.

The present disclosure includes many aspects and features. Moreover, while many aspects and features relate to, and are described in the context of a storage system for biological samples in freezing tanks in a nitrogen environment 100, embodiments of the present disclosure are not limited to use only in this context.

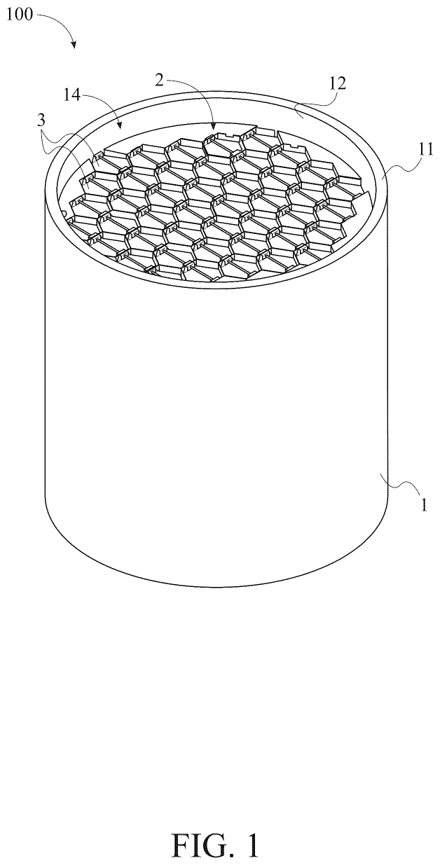

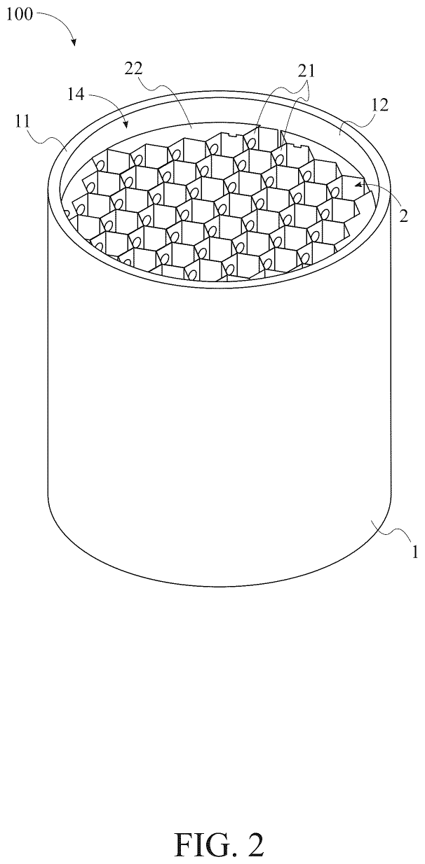

The present invention is a storage system for biological samples in freezing tanks in a nitrogen environment 100, as shown in FIG. 1, comprising a storage tank 1, a frame 2, and a plurality of canisters 3. In the context of the present invention, the storage tank 1 is a vessel to house biological samples, containing liquid and gaseous nitrogen to preserve said biological samples. In the preferred embodiment of the present invention, the storage tank 1 comprises a rim 11, an interior wall 12, a base, and a basin 14. In the context of the present invention, the rim 11 forms the topmost plane of the storage tank 1 and the basin 14 is formed by the volume encapsulated by the interior wall 12, the base, and the plane formed by the rim 11 of the storage tank 1. In the context of the present invention, the base of the storage tank 1 is the bottom plane of the basin 14. In the preferred embodiment of the present invention, the basin 14 is a cylindrical cavity whereby the interior wall 12 composes the circumference of said basin 14. In the preferred embodiment of the present invention, the frame 2 is configured within the basin 14 of the storage tank 1 such that the frame 2 composes a matrix by which the plurality of canisters 3 is removably inserted into the frame 2, as shown in FIG. 1 and FIG. 2. Moreover, in the preferred embodiment of the present invention, the frame 2 is offset below the plane formed by the rim 11. In such embodiments, the offset is sufficient such that a canister 3, when removed, may be placed on the frame 2 and remain below the rim 11. By remaining below the rim 11, biological samples contained within the canisters 3 remain preserved.

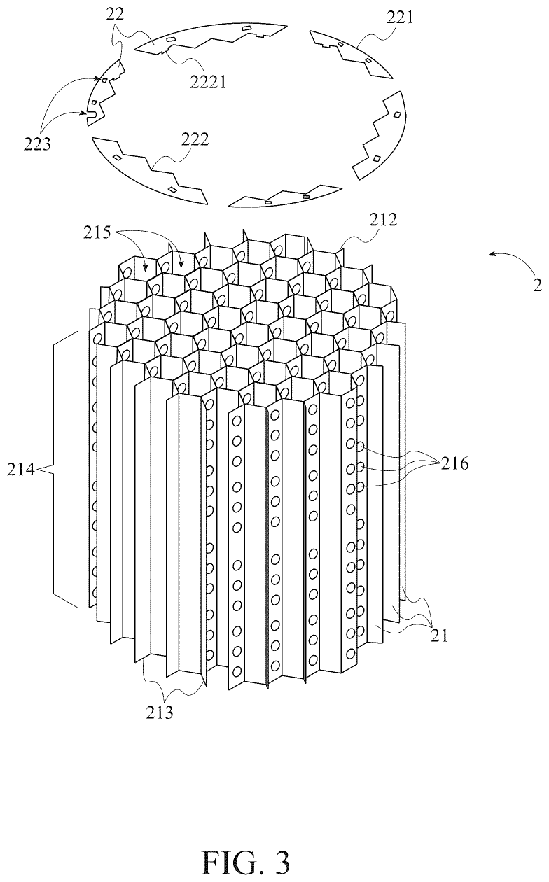

As shown in FIG. 2 and FIG. 3, the frame 2 of the present invention comprises a plurality of corrugated panels 21, and a plurality of end caps 22. In the preferred embodiment of the present invention, plurality of end caps 22 couple to the topmost portion of the plurality of corrugated panels 21, adjacent to the interior wall 12 of the storage tank 1. Furthermore, in the preferred embodiment, the plurality of corrugated panels 21 are arranged such that the corrugated panels 21 compose a plurality of columns 215, similarly to a honeycomb structure. In the preferred embodiment of the present invention, each of the columns 215 comprise a uniform geometric shape. In the preferred embodiment of the present invention, the geometric shape comprised by the columns 215 is a hexagon. In the context of the present invention, each of the plurality of canisters 3 are removably disposed within each of the columns 215. In the preferred embodiment of the present invention, the plurality of end caps 22 are arranged around the interior wall 12 of the storage tank 1 wherein the column 215 produced by the intersection of the wall and the adjacent corrugated panel 21 is insufficient in size to receive a canister 3.

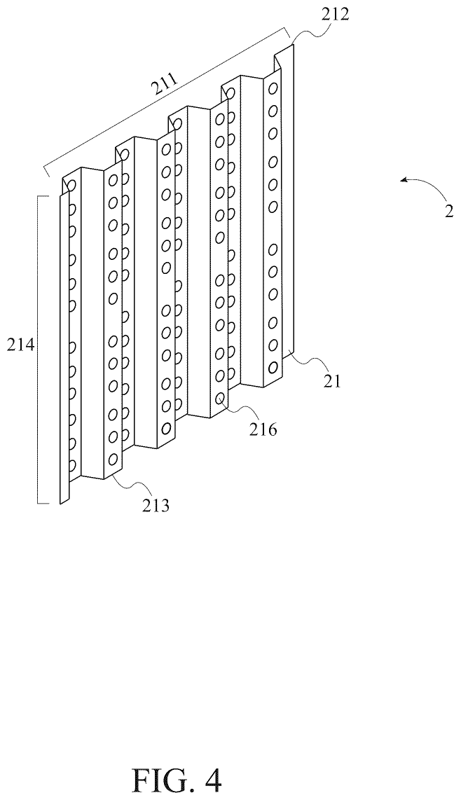

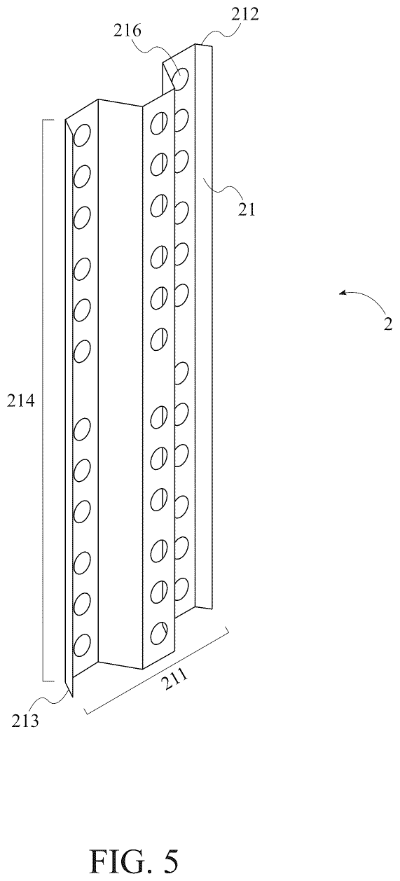

As shown in FIG. 4 and FIG. 5, each of the corrugated panels 21 comprise a length 211, a top edge 212, a bottom edge 213, a height 214, and a plurality of holes 216. In the context of the present invention, the length 211 of the corrugated panel 21 is defined as the horizontal measurement across said corrugated panel 21, wherein the height 214 of the corrugated panel 21 is the vertical measurement. In the context of the present invention, the height 214 of each of the corrugated panels 21 are uniform. As it is understood that the length of a cross section of a circle varies depending on the location of the section being measured, and the basin 14 is a circular cross section, each of the corrugated panels 21 comprise a unique length dependent on a predetermined location within the storage tank 1, as shown by the varying embodiments shown in FIG. 4 and FIG. 5. In the context of the present invention, the plurality of holes 216 traverse through each of the corrugated panels 21. In such embodiments wherein the plurality of corrugated panels 21 comprises a plurality of holes 216, said plurality of holes 216 enable fluid to circulate through the corrugated panels 21 freely, thus sufficiently cooling the biological samples stored within the canisters 3. Additionally, in the context of the present invention, the top edge 212 and the bottom edge 213 are opposing parallel edges, forming the topmost edge and the bottom most edge of each corrugated panel 21, respectively, wherein the bottom edge 213 is positioned on the base of the storage tank 1 and the top edge 212 resides below the rim 11 of the storage tank 1. Moreover, in the preferred embodiment of the present invention, the arrangement of the plurality of corrugated panels 21 within the storage tank 1 creates a rigid structure wherein a user may utilize the collective top edges of each of the corrugated panels 21 as a workspace, without risk of the corrugated panels 21 falling over or collapsing.

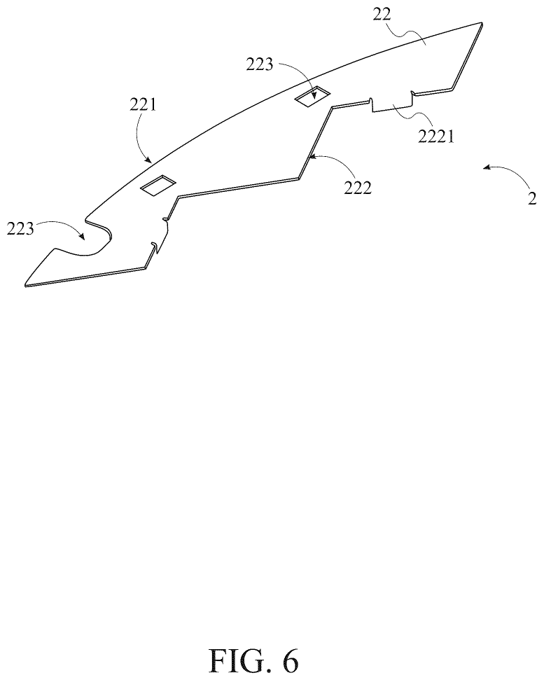

As shown in FIG. 6, in the preferred embodiment of the present invention, each of the end caps 22 comprise an exterior edge 221 and an interior edge 222. In the context of the present invention, the exterior edge 221 and the interior edge 222 are opposing edges of each end cap 22 wherein the exterior edge 221 is positioned adjacent to the interior wall 12 of the storage tank 1. Furthermore, in the preferred embodiment of the present invention, the interior edge 222 is cut out such that said interior edge 222 aligns with adjacent column 215 geometric shape. Moreover, in some embodiments of the present invention, the interior edge 222 of a respective end cap 22 comprises a tab 2221, wherein said tab 2221 secures the end cap 22 to an adjacent corrugated panel 21. In some embodiments of the present invention, an end cap 22 comprises an at least one cutout 223. In such embodiments, the at least one cutout 223 may provide a channel to run wires and perform other utilitarian purposes.

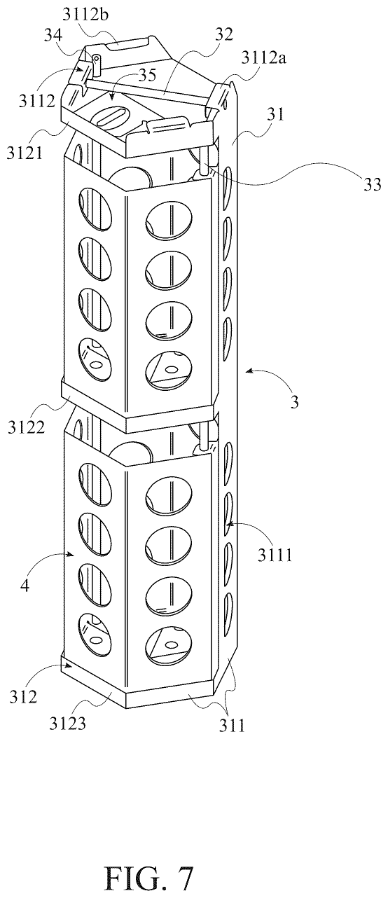

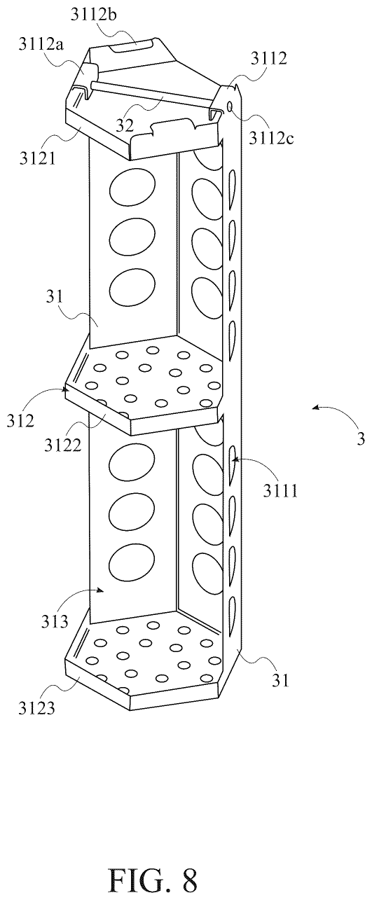

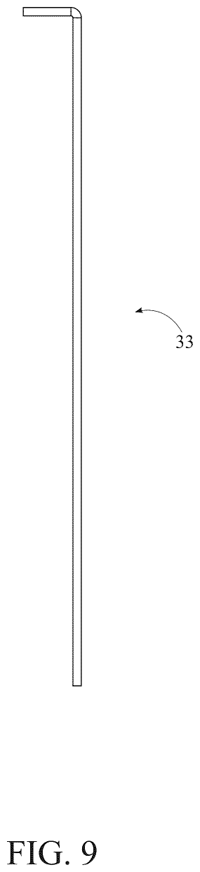

As shown in FIG. 7, in the context of the present invention, each canister 3 is an elongated container 31 comprising the shape of a geometric prism wherein said geometric shape comprising the cross section is corresponsive to the geometric shape of the plurality of columns 215 composed by the plurality of corrugated panels 21. As further shown in FIG. 7 and FIG. 8, each canister 3 comprises a plurality of side walls 311, a plurality of platforms 312, an at least one recess 313, a pull bar 32, a guide axel 33, and a locking rod 34. In the context of the present invention, the plurality of side walls 311 extends vertically, composing the geometric shape of the canister 3. Furthermore, in the context of the present invention, the plurality of platforms 312 extend normally inward from the plurality of side walls 311 such that each is platform 312 of each respective canister 3 is vertically offset along the plurality of side walls 311; the at least one recess 313 being formed by the volume composed by the plurality of side walls 311 and adjacent platforms 312. Additionally, in the preferred embodiment of the present invention, each canister 3 further comprises an at least one receptacle 4, wherein said at least one receptacle 4 is interposed within the at least one recess 313. Furthermore, in the preferred embodiment of the present invention, the pull bar 32 is configured horizontally interposed between the plurality of side walls 311, offset above the topmost platform 3121 of the plurality of platforms 312, such that the pull bar 32 is supported by the plurality of side walls 311. Further, in the preferred embodiment of the present invention, each respective guide axel 33, as shown in FIG. 9, vertically extends through each respective canister 3 and respective receptacle 4 such that each respective receptacle 4 hingedly rotates about the guide axel 33, thus enabling a user to selectively access each respective receptacle 4. Furthermore, in the preferred embodiment of the present invention, each locking rod 34 is removably inserted through each of the respective canisters 3 such that when inserted through said canister 3, the locking rod 34 prevents access to the respective receptacle 4 by preventing said receptacle 4 from rotating about the guide axel 33. In some embodiments of the present invention, each canister 3 further comprises a unique identifier 35, thus enabling a user to properly identify each of the canisters 3.

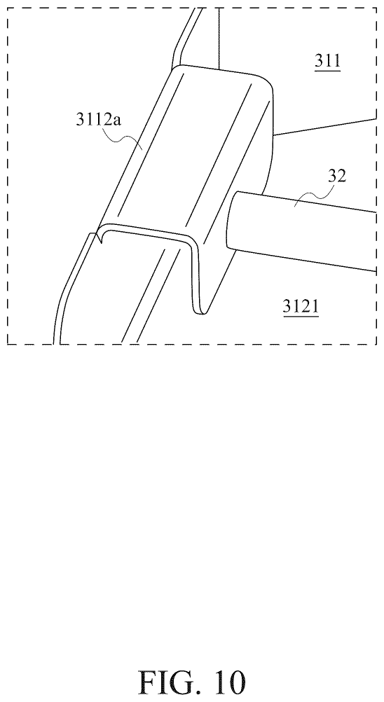

In the preferred embodiment of the present invention, the plurality of platforms 312 comprises a topmost platform 3121, a median platform 3122, and a base platform 3123, thus composing a plurality of recesses 313, numbering two recesses interposed between the topmost platform 3121 and the median platform 3122, and the median platform 3122 and the base platform 3123. In such embodiments, a plurality of receptacles 4 numbering two receptacles 4, are integrated into the canister 3 such that each of the receptacles 4 are hingedly interposed within each of the respective recesses 313. Furthermore, in the preferred embodiment of the present invention, the plurality of side walls 311 and plurality of platforms 312 of each canister 3 comprise a plurality of holes 3111 such that fluid, such as liquid or gaseous nitrogen, may freely flow through the plurality of holes. Additionally, within the preferred embodiment of the present invention, the plurality of side walls 311 comprise a plurality of tabs 3112 positioned above the topmost platform 3121. In some embodiments of the present invention, the plurality of tabs 3112 provides a structural support 3112b for the plurality of walls 311, thereby inhibiting said walls 311 from bending. In some embodiments of the present invention, the plurality of tabs 3112 comprises L-shaped tabs 3112a such that said L-shaped tabs 3112a support the pull bar 32. In such embodiments of the present invention wherein the L-shaped tabs 3112a support the pull bar 32, the pull bar 32 traverses between two L-shaped tabs 3112a positioned on opposing side walls 311, through a plurality of holes 3112c comprised by said L-shaped tabs 3112a, as shown in FIG. 10.

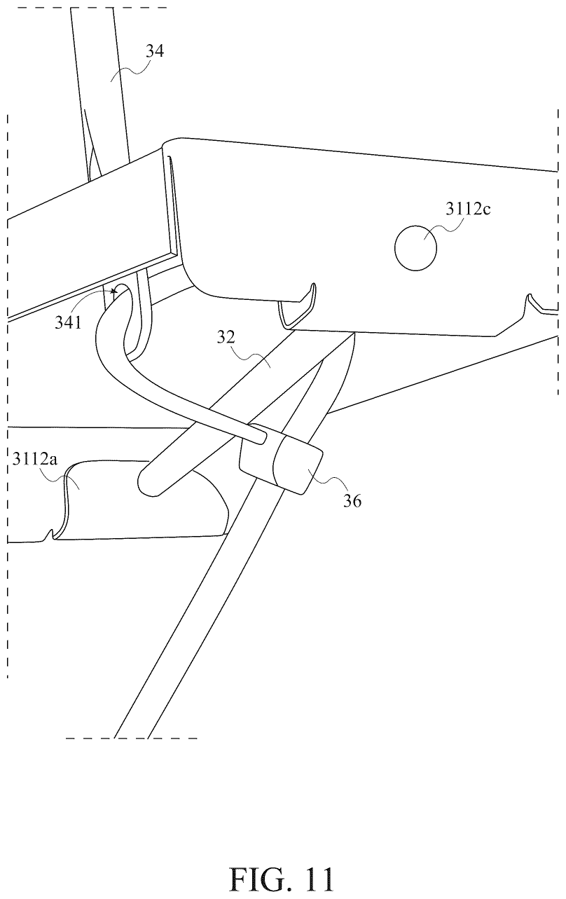

Moreover, as shown in FIG. 11, in the preferred embodiment of the present invention, the canister 3 further comprises a locking mechanism 36 wherein the locking mechanism 36 secures the locking rod 34 to the canister 3. Additionally, within the preferred embodiment of the present invention, the locking rod 34 comprises a through hole 341 wherein said locking mechanism 36 traverses, thus securing the locking rod 34 to the pull bar 32. In the context of the present invention, the locking mechanism 36 is a locking mechanism 36, such as a cable tie mechanism, comprising a static construction, as the low temperatures within the storage tank 1 would inhibit the use of traditional padlock designs, and those designs similar, as the cylinders and components within such traditional lock designs would freeze, thus being rendered inoperable. In such cases, allowing the traditional locks to reach a temperature in which they would dethaw would subsequently allow the samples stored within the canisters 3 to reach the same temperature, thereby risking the preservation of such samples.

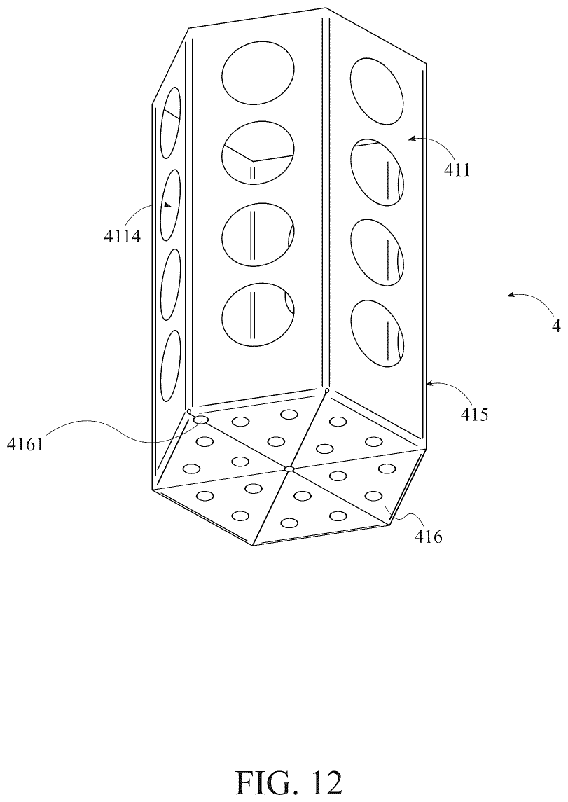

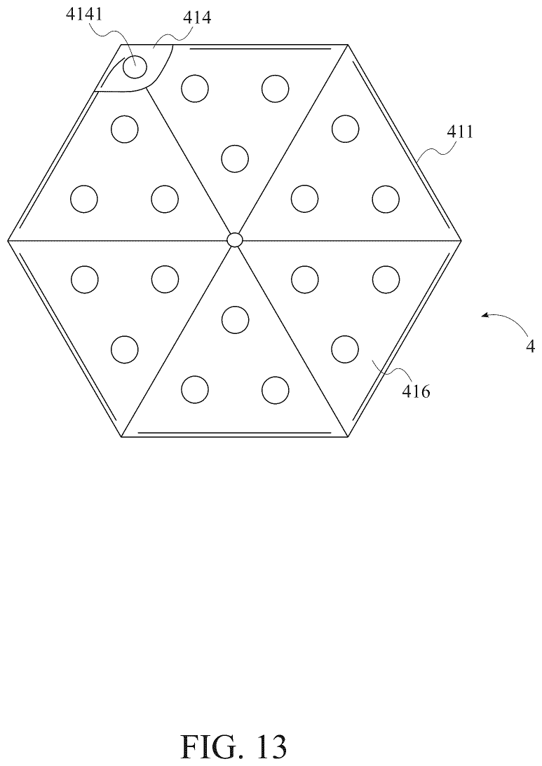

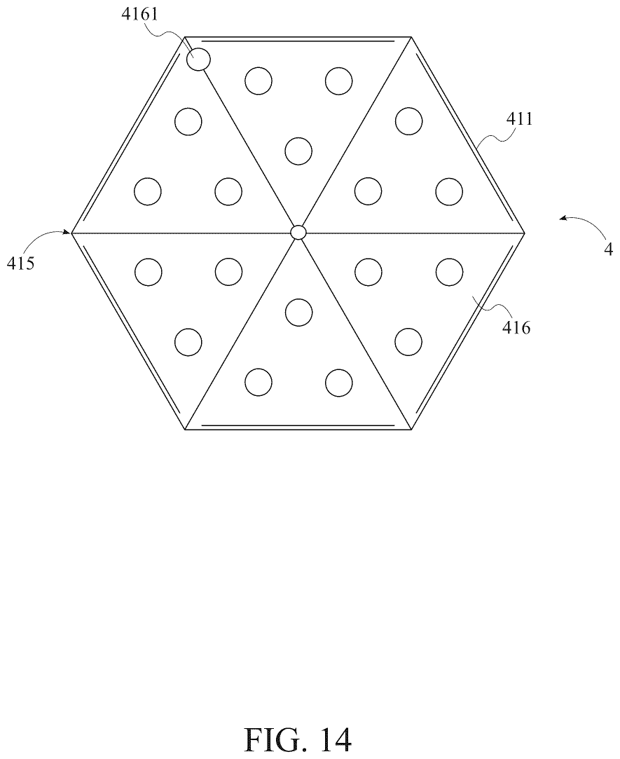

As shown in FIG. 7, FIG. 12, FIG. 13, and FIG. 14, each receptacle 4 is a vessel comprising a plurality of side walls 411, a base 416, and a tab 414. In the context of the present invention, the plurality of side walls 411 extend normally upward from the base 416. Additionally, the tab 414 extends normally inward from one of the side walls 411, parallel to the base 416, whereby said tab 414 comprises a guide hole 4141, as shown in

FIG. 13. Furthermore, in the preferred embodiment of the present invention, the base 416 further comprises a guide hole 4161, as shown in FIG. 14, wherein the guide axel 33, when the receptacle 4 is integrated into the respective canister 3, traverses the guide holes 4141,4161 of the tab 414 and the base 416. Furthermore, in the preferred embodiment of the present invention, each receptacle 4 is a geometric prism shaped vessel further comprising a plurality of corners 415.

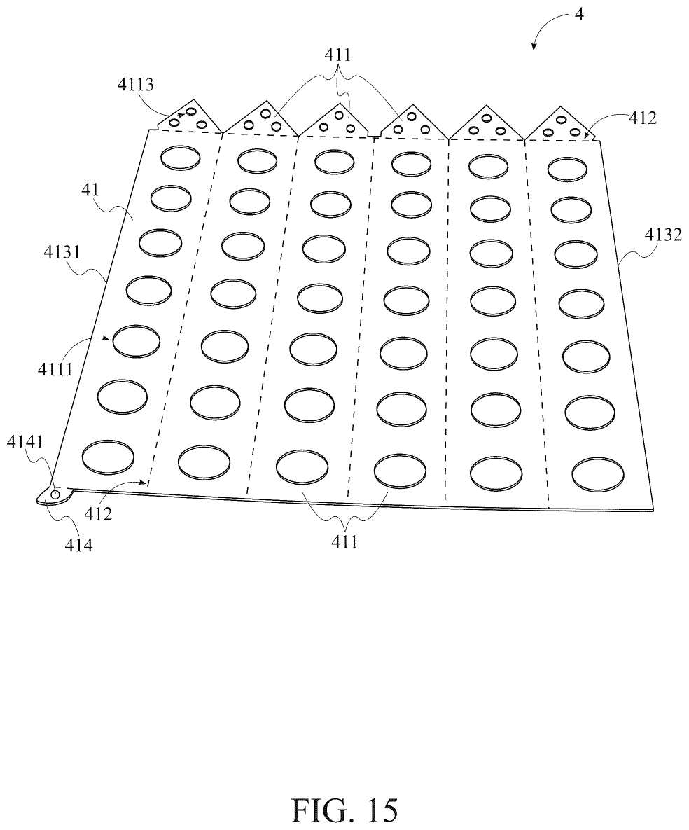

In the preferred embodiment of the present invention, as shown in FIG. 15, each of the receptacles 4 are composed of a metal sheet 41 divided into a plurality of sections 411 wherein said sections 411 are separated by a plurality of fold lines 412. In the context of the present invention, the plurality of fold lines 412 are imaginary lines wherein the sheet 41 is folded to compose the plurality of corners 415. Furthermore, each of the sections further comprise a base panel 4112, wherein the preferred embodiment, each base panel 4112 is a triangular panel. In the preferred embodiment of the present invention, the base panels 4112 are separated from the sections 411 by the fold lines 412 whereby said fold lines 412 separating the base panels 4112 and the sections 411 are perpendicular to those fold lines 412 separating the plurality of sections 411. Moreover, in the preferred embodiment of the present invention, plurality of sections 411 comprises a plurality of holes 4111 and each of the base panels 4112 comprise a plurality of holes 4113. The sheet 41 further comprises a plurality of lateral edges, a first lateral edge 4131 and a second lateral edge 4132, whereby said sheet 41 is bent into a geometric prism such that the plurality of sections 411 composes the plurality of side walls 411, and the plurality of lateral edges 4131,4132 are joined together. Additionally, in the preferred embodiment of the present invention, each of the base panels 4112 are bent perpendicularly to the adjacent section 411, thus composing the base 416 of the receptacle 4. Moreover, the sheet 41 further comprises the tab 414, wherein said tab 414 is bent parallel to the base 416.

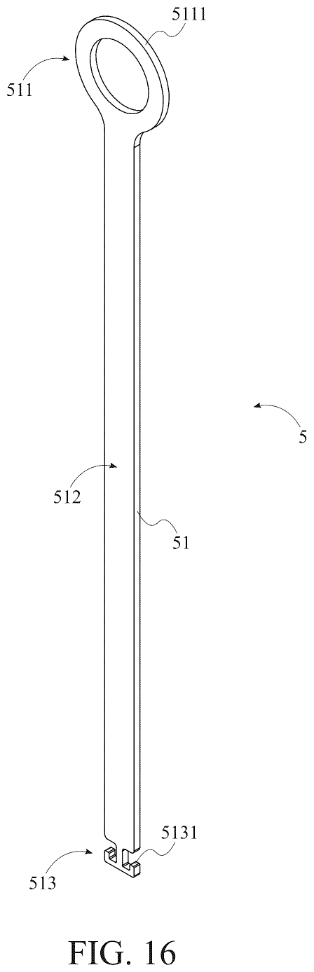

In some embodiments of the present invention, as shown in FIG. 16, the storage system for biological samples in freezing tanks in a nitrogen environment 100 further comprises a canister removal mechanism 5 wherein said canister removal mechanism 5 removably attaches to the pull bar 32 of the canister 3 to facilitate the removal and installation of the plurality of canisters 3 in respect to each respective column 215. In the preferred embodiment of the present invention, the canister removal mechanism 5 comprises an elongated member 51, a handle 5111, and an at least one hook 5131 wherein the elongated member 51 comprises a first distal end 511 and a second distal end 513 such that said distal ends 511,513 are opposing ends of the elongated member 51. Furthermore, in the context of the present invention, the portion of the elongated member 51 is referred to herein as the proximal portion 512. Further, the handle 5111 is located at the first distal end 511 whereby the at least one hook 5131 is disposed at the second distal end 513, and the at least one hook 5131 removably latches onto the pull bar 32 of each canister 3.

Although the invention has been explained in relation to its preferred embodiment, it is to be understood that many other possible modifications and variations can be made without departing from the spirit and scope of the invention.

What is claimed is:

1. A storage system for biological samples in freezing tanks in a nitrogen environment comprising:
a frame; and
a plurality of canisters;
wherein:
the frame comprises a plurality of corrugated panels arranged within a storage tank such that the corrugated panels compose a plurality of uniform geometrically shaped columns;
each canister of the plurality of canisters being a geometric prism shaped container, corresponding with the shape of the columns of the frame such that each canister is slidably housed within a respective column;
each canister comprising an at least one receptacle wherein said at least one receptacle is hingedly integrated into the respective canister;
each canister further comprising a pull bar, a guide axel, and a locking rod;
each guide axel extending through a respective canister such that the at least one receptacle of said respective canister hingedly rotates about said respective guide rod;
each locking rod adjustably traversing through the respective canister and at least one receptable, such that when engaged, the locking rod prevents access to the respective at least one receptacle; and
each pull bar being arranged horizontally at a topmost portion of the respective canister.

2. The storage system for biological samples in freezing tanks in a nitrogen environment as claimed in claim 1, wherein each of the canisters further comprises:
a plurality of side walls;
a plurality of platforms; and
a plurality of recesses;
wherein:
the plurality of side walls being the outermost panels of each respective canister, wherein said plurality of side walls extend vertically along the canister;
the plurality of platforms extending horizontally inward from the plurality of side walls of each respective canister, wherein each platform of the plurality of platforms of the respective canister is vertically offset from the adjacent platform;
the plurality of recesses are cavities interposed between adjacent platforms, wherein the at least one receptacle is interposed within at least one of the plurality of recesses of the respective canister.

3. The storage system for biological samples in freezing tanks in a nitrogen environment as claimed in claim 2, wherein the plurality of side walls of each canister comprise a plurality of tabs;
wherein:
the plurality of tabs comprise a plurality of L-shaped tabs extending inwardly from the topmost portion of the plurality of side walls of the respective canister; and
the pull bar extending between the plurality of L-shaped tabs, such that the plurality of L-shaped tabs suspends said pull bar.

4. The storage system for biological samples in freezing tanks in a nitrogen environment as claimed in claim 2, wherein:
each receptacle comprises:
a plurality of side walls;
a base; and
a tab;
wherein:
the plurality of side walls of the receptacle extending normally vertical from the base;
the tab extending normally inward from a topmost portion of one side wall of the plurality of side walls of each respective receptacle; and
the guide rod traversing through the tab and the base of each respective receptacle.

5. The storage system for biological samples in freezing tanks in a nitrogen environment, as claimed in claim 4, wherein the plurality of side walls of the canister and the plurality of side walls of the receptacles each comprise a plurality of holes.

6. The storage system for biological samples in freezing tanks in a nitrogen environment, as claimed in claim 5, wherein the base of each receptacle and the tab of each receptacle comprise a guide hole wherein the guide rod traverses through the guide holes of each of the base and the tab of the respective receptacle.

7. The storage system for biological samples in freezing tanks in a nitrogen environment as claimed in claim 4 wherein:
each receptacle is composed of:
a sheet comprising:
a plurality of sections;
a plurality of base panels;
a plurality of lateral edges; and
a tab;
wherein:
the sheet is folded along a plurality of fold lines, wherein said fold lines separate the plurality of sections, thus forming the plurality of side walls of the receptacle;
the plurality of lateral edges are conjoined together thus forming an enclosed shape; and
the plurality of base panels is folded perpendicularly inward, thus forming the base of the receptacle.

8. The storage system for biological samples in freezing tanks in a nitrogen environment, as claimed in claim 1, wherein each of: the frame, the plurality of canisters, and the at least one receptacle; comprise a plurality of holes.

9. The storage system for biological samples in freezing tanks in a nitrogen environment, as claimed in claim 7 wherein the plurality of base panels are triangular and comprise an at least one through hole.

10. The storage system for biological samples in freezing tanks in a nitrogen environment, as claimed in claim 1, further comprising a locking mechanism wherein said locking mechanism secures the locking rod in a fixed location.

11. The storage system for biological samples in freezing tanks in a nitrogen environment, as claimed in claim 1, wherein the frame further comprises a plurality of end caps;
said end caps extend over a topmost portion of the frame.

12. The storage system for biological samples in freezing tanks in a nitrogen environment, as claimed in claim 11, wherein the storage tank comprises:
a rim;
an interior wall; and
a basin;
wherein:
the rim forms a top-most plane of the storage tank;
the interior wall is a circumferential wall forming the basin; and
the frame is positioned in the basin of the storage tank such that the frame is entirely offset below the rim.

13. The storage system for biological samples in freezing tanks in a nitrogen environment, as claimed in claim 12, wherein each of the end caps comprise:
an interior edge;
an exterior edge; and
a plurality of cutouts;
wherein:
each of the end caps are panels wherein the exterior edge engages the circumferential portion of the interior wall of the cyrogenic tank and the interior edge is formed to configure to the shape of the columns; and
the plurality of cutouts are channels through the respective end cap.

14. A storage system for biological samples in freezing tanks in a nitrogen environment, comprising:
a storage tank;
a frame;
a plurality of canisters; and
an at least one receptacle;
wherein:
the storage tank comprises a basin;
the frame is disposed within the basin of the storage tank;
the frame is comprises a plurality of corrugated panels arranged such that said corrugated panels compose a plurality of columns;
each canister of the plurality of canisters is a geometrically shaed tubular container whereby each canister is removably integrated into the columns of the frame;
the at least one receptacle housed within one of the canisters of the plurality of canisters;
the at least one receptacle is hingedly coupled to the respective canister such that the adjustably accessible;
each of the canisters comprise:
a plurality of platforms;
a pull bar;
a guide axel;
a locking rod; and
a lock;
wherein:
the plurality of platforms support the at least one receptacle within the respective canister;
the guide axel traverses through each canister and respective receptacle such that said receptacle rotates about said guide axel;
the locking rod removably traversing through each canister and the respective receptacle, prohibiting rotation of said receptacle when inserted; and
the lock securing the locking rod to the canister.

15. The storage system for biological samples in freezing tanks in a nitrogen environment, as claimed in claim 14, wherein the storage tank comprises:
a rim;
an interior wall; and
a basin;
wherein:
the rim forms a top-most plane of the storage tank;
the interior wall is a circumferential wall forming the basin; and
the frame is positioned in the basin of the storage tank such that the frame is entirely offset below the rim.

16. The storage system for biological samples in freezing tanks in a nitrogen environment, as claimed in claim 15, wherein the frame further comprises:
a plurality of end caps wherein each end cap comprises an exterior edge;
wherein:
each of the corrugated panels comprise:
a plurality of through holes; and
a top edge;
each of the end caps couple to at least one of the corrugated panels wherein said end caps are located at the top edge of the respective corrugated panel; and
the exterior edge of each end cap is positioned against the interior wall of the storage tank.

17. The storage system for biological samples in freezing tanks in a nitrogen environment, as claimed in claim 16, wherein each canister further comprises:
a plurality of side walls; and
a plurality of tabs;
wherein:
the plurality of side walls of each canister comprises a plurality of holes;
the plurality of tabs are positioned at a topmost portion of the plurality of side walls, extending inwardly; and
the plurality of tabs supporting the pull tab.

18. The storage system for biological samples in freezing tanks in a nitrogen environment, as claimed in claim 17, wherein each of the receptacles comprises:
a plurality of side walls; and
a base;
wherein:
the plurality of side walls comprise a plurality of holes and a tab;
the tab comprises a guide hole;
the base comprises a plurality of holes and a guide hole
each guide axel traverses the respective canister and each respective receptacle, through the respective guide holes of the tab and base.

19. A storage system for biological samples in freezing tanks in a nitrogen environment, comprising:
a storage tank;
a frame;
a plurality of canisters;
an at least one receptacle; and
a canister removal mechanism;
wherein:
the storage tank comprises a basin;
the frame is disposed within the basin of the storage tank;
the frame is comprises a plurality of corrugated panels arranged such that said corrugated panels compose a plurality of columns;
each canister of the plurality of canisters is a geometrically shaed tubular container whereby each canister is removably integrated into the columns of the frame;
the at least one receptacle housed within one of the canisters of the plurality of canisters;
the at least one receptacle is hingedly coupled to the respective canister such that the adjustably accessible;
each of the canisters comprise:
a plurality of platforms;
a pull bar;
a guide axel;
a locking rod; and
a lock;
wherein:
the plurality of platforms support the at least one receptacle within the respective canister;
the guide axel traverses through each canister and respective receptacle such that said receptacle rotates about said guide axel;
the locking rod removably traversing through each canister and the respective receptacle, prohibiting rotation of said receptacle when inserted; and
the lock securing the locking rod to the canister;
the canister removal mechanism removably latches onto the pull bar, thus facilitating the removal and insertion of each respective canister into the respective column.

20. The storage system for biological samples in freezing tanks in a nitrogen environment, as claimed in claim 19, wherein the canister removal mechanism comprises:
an elongated member;
a handle; and
an at least one hook;
wherein:
the elongated member comprises a first distal end and a second distal end such that said distal ends are opposing ends of the elongated member;
the handle is disposed at the first distal end;
the at least one hook is disposed at the second distal end; and
the at least one hook latches onto the pull bar of each canister.
